(19) United States
(12) Patent Application Publication
(10) Pub. No.: US 2016/0000554 A1
(43) Pub. Date: Jan. 7, 2016

(54) APPARATUS AND METHOD FOR REGENERATION OF LIGAMENTS AND TENDONS

(71) Applicant: UNIVERSITY OF PITTSBURGH - OF THE COMMONWEALTH SYSTEM OF HIGHER EDUCATION, Pittsburgh, PA (US)

(72) Inventors: Savio L-Y. Woo, Pittsburgh, PA (US); Katie Farraro, Pittsburgh, PA (US); Andrea Speziali, Pittsburgh, PA (US); Kwan Eun Kim, Pittsburgh, PA (US); Patrick J. McMahon, Pittsburgh, PA (US)

(21) Appl. No.: 14/772,670

(22) PCT Filed: Mar. 6, 2014
(86) PCT No.: PCT/US2014/021377

§ 371 Date: Sep. 3, 2015

Related U.S. Application Data
(60) Provisional application No. 61/773,435, filed on Mar. 6, 2013.

(51) Int. Cl. A61F 2/08; A61B 17/11; A61B 17/04

(52) U.S. Cl. CPC A61F 2/0811; A61B 17/0401; A61B 17/1146; A61F 2002/0882; A61F 2002/0847; A61F 2002/0817; A61F 2210/0004; A61F 2220/0016; A61F 2230/0067; A61F 2230/0069; A61B 2017/0414; A61B 2017/0464; A61B 2017/00004

(57) ABSTRACT

A method for repairing a ligament or tendon, the method comprising: passing one or more sutures through and/or around each respective end of a completely or partially torn ligament or tendon; placing a generally cylindrical hollow body or ring over the respective ends of the completely or partially torn ligament or tendon; passing the one or more sutures through the generally cylindrical hollow body or ring; and tying the one or more sutures around or through the generally cylindrical hollow body or ring wherein the sutures are secured in place around or through the generally cylindrical hollow body or ring via one or more notches or holes defined by the generally cylindrical hollow body or ring. The method may further comprise tensioning the one or more sutures while tying them around or through the generally cylindrical hollow body or ring so that the respective ends of the completely or partially torn ligament or tendon are brought into and stay in contact with each other inside the generally cylindrical hollow body or ring after the tying is complete. The generally cylindrical hollow body or ring preferably is biodegradable and may be coated with an ECM.

### RELATED APPLICATIONS

This application claims priority benefit under 35 U.S.C. §119(e) of U.S. Provisional Application No. 61/773,435, filed Mar. 6, 2013, entitled Metallic and Extracellular Matrix Bioscaffolds for Regeneration of the Anterior Cruciate Ligament, the contents of which are herein incorporated by reference.

### GOVERNMENTAL RIGHTS

This invention was made with government support under Grant Number 0812348 awarded by the United States National Science Foundation. The government has certain rights in the invention.

### FIELD

The present disclosure relates to devices, systems and methods for repair of ligaments and tendons such as the anterior cruciate ligament (ACL).

### BACKGROUND INFORMATION

The ACL is the most frequently injured knee ligament with tears occurring in well over 200,000 people in the U.S. each year. Midsubstance ACL ruptures have a limited capacity for healing, so ACL reconstruction using soft tissue autografts has become the clinical gold standard of treatment. However, though this treatment relieves pain and can help maintain knee stability in the short-term, long-term follow-up studies (10+ years) of these patients showed up to 25% unsatisfactory results, including osteoarthritis, residual pain, and donor site morbidity.

These complications have led to alternative clinical and experimental approaches to heal the ACL. A successfully regenerated ACL would have many advantages over surgical reconstruction as its complex anatomical features can be preserved, while complications, including graft donor site morbidity and procedural complexity, could be reduced or eliminated.

Laboratory research has shown that healing of partial ACL tears can be stimulated in animal models through the use of scaffolds and/or growth factors, e.g. bFGF, hyaluronic acid, bone marrow-derived mesenchymal stem cells, and platelet-rich plasma. However, all treated ACLs remained quite abnormal in terms of their morphology and biomechanical properties.

A bioscaffold small intestinal submucosa (SIS) and an Extracellular Matrix (ECM) sheet in combination with its 3-D hydrogel form was used to promote ACL healing following suture repair in a goat model. To reduce joint instability, a bone-to-bone fixation method (suture augmentation) was used to restore initial joint stability. Both SIS treatment and augmentation techniques showed improvements in healing over suture repair alone, but structural properties of the femur-ACL-tibia complex and joint stability remained inferior to the normal ACL. Preferably, healing would be improved by partial loading of the ACL at time zero to avoid disuse atrophy and degradation of the insertion sites.

Thus, there remains a considerable need for an apparatus and method for repairing a ligament or tendon, such as the ACL, that allows for partial loading of the ligament or tendon at time zero to avoid disuse atrophy and degradation of the insertion sites to produce a regenerated ligament or tendon that functions virtually as well as the normal ligament or tendon.

### SUMMARY OF THE DISCLOSURE

In a preferred aspect, the present disclosure is directed to a method for repairing a ligament or tendon, the method comprising: passing one or more sutures through and/or around each respective end of a completely or partially torn ligament or tendon; placing a generally cylindrical hollow body or ring over the respective ends of the completely or partially torn ligament or tendon; passing the one or more sutures through the generally cylindrical hollow body or ring; and tying the one or more sutures around or through the generally cylindrical hollow body or ring wherein the sutures are secured in place around or through the generally cylindrical hollow body or ring via one or more notches or holes defined by the generally cylindrical hollow body or ring.

In another preferred aspect, the method may further comprise tensioning the one or more sutures while tying them around or through the generally cylindrical hollow body or ring.

In a further preferred aspect, the method may further comprise tensioning the one or more sutures while tying them around or through the generally cylindrical hollow body or ring so that the respective ends of the completely or partially torn ligament or tendon are brought into and stay in contact with each other inside the generally cylindrical hollow body or ring after the tying is complete.

In another preferred aspect, the method may further comprise applying an ECM to the generally cylindrical hollow body or ring.

In a further preferred aspect, the generally cylindrical hollow body or ring may comprise a biodegradable material.

In another preferred aspect, the generally cylindrical hollow body or ring may comprise a biodegradable material having an in-situ degradation rate consistent with a healing time of the ligament or tendon.

In a further preferred aspect, the generally cylindrical hollow body or ring may comprise magnesium or a magnesium alloy.

In another preferred aspect, the completely or partially torn ligament may comprise an ACL.

In an additional preferred aspect, the method may further comprise providing suture augmentation to a joint which the ACL is a part of by passing one or more sutures through each of tibial and femoral bone tunnels disposed adjacent to the joint and securing the sutures to the joint under tension.

In another preferred aspect, the generally cylindrical hollow body or ring may have a first end diameter smaller than a second end diameter and is tapered therebetween.

In another preferred aspect, the present disclosure is directed to an implant device for use in surgically repairing a ligament or tendon, comprising: a generally cylindrical hollow body, wherein the generally cylindrical hollow body defines a plurality of holes and/or notches for tensioning or tying sutures around or through the generally cylindrical hollow body. Such implant device may further comprise an ECM.

In another preferred aspect, the generally cylindrical hollow body of the implant device may comprise a biodegradable material.

In a further preferred aspect, the generally cylindrical hollow body of the implant device may comprise a biodegradable material having an in-situ degradation rate consistent with a healing time of the ligament or tendon.

In another preferred aspect, the generally cylindrical hollow body of the implant device may comprise magnesium or a magnesium alloy.

In a further preferred aspect, the generally cylindrical hollow body of the implant device may have a first end diameter smaller than a second end diameter and is tapered therebetween.

### BRIEF DESCRIPTION OF THE DRAWINGS

For the present disclosure to be easily understood and readily practiced, the disclosure will now be described, for the purposes of illustration and not limitation, in conjunction with the following figures, wherein:

FIG. 1 is a perspective view of a preferred embodiment of an implantable cylinder or ring for use in accordance with a preferred method for repairing a ligament or tendon according to the present disclosure;

FIG. 2 is a perspective view of another preferred embodiment of an implantable cylinder or ring for use in accordance with a preferred method for repairing a ligament or tendon according to the present disclosure;

FIG. 3 is a perspective view of yet a further preferred embodiment of an implantable cylinder or ring for use in accordance with a preferred method for repairing a ligament or tendon according to the present disclosure;

FIG. 4 is a perspective view of another preferred embodiment of an implantable cylinder or ring for use in accordance with a preferred method for repairing a ligament or tendon according to the present disclosure;

FIG. 5 is a perspective view of yet a further preferred embodiment of an implantable cylinder or ring for use in accordance with a preferred method for repairing a ligament or tendon according to the present disclosure;

FIGS. 6-9 are schematic views of a preferred method for repairing a ligament or tendon such as an ACL according to the present disclosure;

FIGS. 10A-10B show additional side plan views of the preferred embodiment shown in FIG. 4 of an implantable cylinder or ring for use in accordance with a preferred method for repairing a ligament or tendon according to the present disclosure; and

FIGS. 11-12 are schematic views of another preferred method for repairing a ligament or tendon such as an ACL according to the present disclosure.

### DETAILED DESCRIPTION OF PREFERRED EMBODIMENTS

As used herein in the specification and claims, including as used in the examples and unless otherwise expressly specified, all numbers may be read as if prefaced by the word “about”, even if the term does not expressly appear. Also, any numerical range recited herein is intended to include all sub-ranges subsumed therein.

Despite its low healing capacity, it has recently been shown that ACL healing can be stimulated by biological augmentation. An ECM bioscaffold with suture repair according to preferred embodiments and methods of the present disclosure preferably can promote healing of an injured ACL or other ligament or tendon.

Because the process of ACL healing is slow, mechanical augmentation is desirable to restore initial joint stability and allow immediate loading of the joint at time zero to prevent disuse atrophy and degradation of the insertion sites due to a lack of stress. Preferably, a magnesium (Mg)-based ring 10 and suture implantation technique can be used for mechanical augmentation of an injured ACL or other ligament or tendon to promote better healing and improved results.

The present disclosure is directed to using a biodegradable metallic device 10 to partially load the ACL at time zero or other ligament or tendon.

Preferably, the Mg-based ring 10 will bridge the gap 25 between the two torn ends 16 and 18 of a completely or partially ruptured ACL 15. In addition, a preferred suture augmentation technique is employed to restore joint stability while allowing some loading of the healing ligament at time zero to prevent disuse atrophy of the insertion sites.

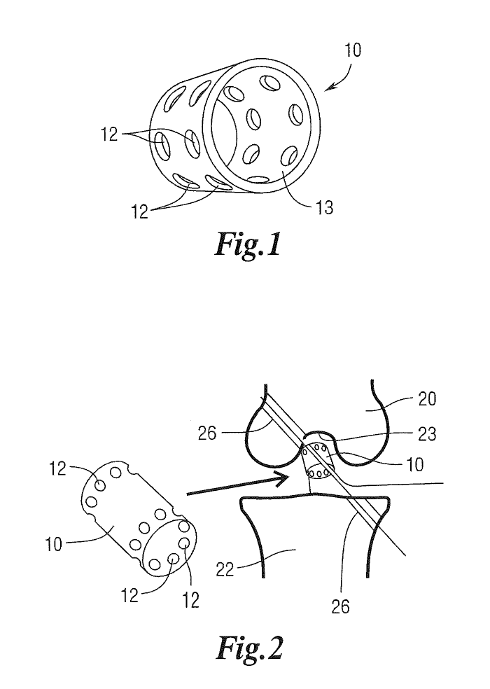

In a first preferred embodiment shown in FIGS. 1 and 2, generally cylindrical hollow body or ring 10 defines one or more suture holes 12 for sutures 17 to pass through in accordance with preferred aspects of the present disclosure. Preferably, suture holes 12 are disposed around the upper and lower circumference as shown. Also, the ring 10 may preferably define a flared tibial end 13. Ring 10 preferably may come in various sizes produced to reflect interspecimen variation and is made of commercial alloy AZ31 which may be specifically designed to have an in-situ degradation rate consistent with ACL healing.

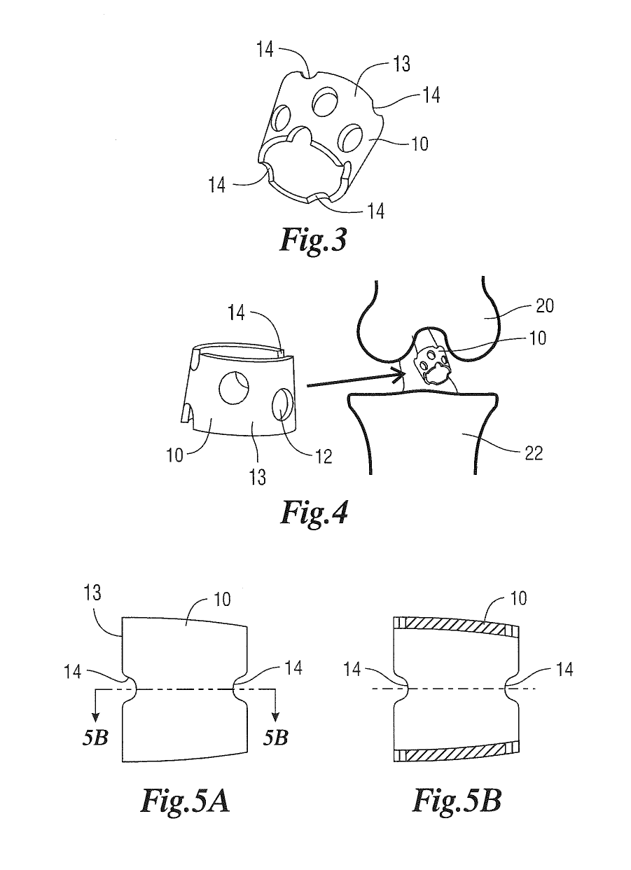

In a second preferred embodiment shown in FIGS. 3 and 5, ring 10 is generally cylindrically shaped and defines one or more suture notches 14 to allow sutures 17 from femoral and tibial stumps 16 and 18, respectively, to be tied around and traverse the length of ring 10 in accordance with preferred aspects of the present disclosure. Preferably, suture notches 14 are disposed around the upper and lower circumference as shown. Also, the ring 10 may preferably define a flared tibial end 13. Ring 10 of this preferred embodiment may come in various sizes produced to reflect interspecimen variation and is made of commercial alloy AZ31 which may be specifically designed to have an in-situ degradation rate consistent with ACL healing.

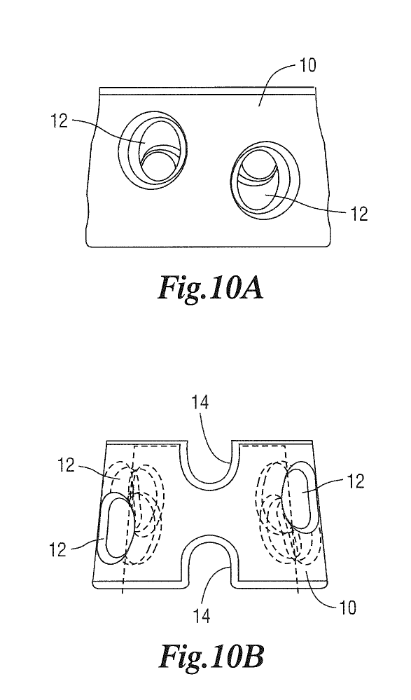

In a third preferred embodiment shown in FIGS. 4 and 10A-10B, ring 10 is generally cylindrically shaped and defines one or more suture holes 12 and suture notches 14 to allow sutures 17 from femoral and tibial stumps 16 and 18, respectively, to be tied through and/or around ring 10 and traverse the length of ring 10 in accordance with preferred aspects of the present disclosure. Preferably, suture notches 14 are disposed around the upper and lower circumference, while suture holes 12 are disposed intermediate thereto as shown. As shown in FIG. 10B, alternate placements of suture holes 12a (shown in dashed lines) preferably may be used depending upon the application. Also, the ring 10 may preferably define a flared tibial end 13. Ring or cylinder 10 of this preferred embodiment may come in various sizes produced to reflect interspecimen variation and is preferably may be made of magnesium or a commercial alloy AZ31 or a single crystal Mg material either of which preferably may be specifically designed to have an in-situ degradation rate consistent with ACL healing. Ring 10 preferably may also be hinged on one longitudinal side and have a clasping or other closure mechanism on an opposite side to accommodate placing the ring over a partially torn ligament or tendon such as an ACL.

Ring 10 preferably has a slightly larger diameter on one end (tibial end 13) and has a geometry and dimensions chosen based on the geometry of the ligament or tendon to be repaired. Along both the top and bottom edges of the ring 10, notches 14 are present in order to hold sutures 17 in place as they are tied from the ligament ends around the Mg-based ring 10 and tied under tension.

Preferably, the surface of the Mg-based alloy ring 10 and/or the ligament or tendon 15 could also be treated with an ECM with or without its 3-D hydrogel form, such as an SIS or a urinary bladder matrix (UBM) applied as a coating or sheet wrapper, to accelerate healing of the ligament or tendon 15. In addition, the hydrogel preferably may be injected into the ligament or tendon 15 instead of being used in a sheet wrapper form. Thus, the system of the present disclosure can serve as a smart, biodegradable complex, capable of integrating with healing tissues and enabling regeneration while the porous metallic scaffold or ring 10 degrades.

Though rings 10 and the preferred methods of the present disclosure maybe used for ACL repair, the application thereof could also be used to bridge the gap 25 between the torn ends of other ligaments and/or tendons for improved healing. The rings 10 and the preferred methods of the present disclosure can provide structural support and limit hypertrophy of healing tissue, leading to better tissue quality in such other ligaments or tendons, where again ring 10 may preferably be used as a scaffold for biological augmentation to accelerate tissue healing, such as in combination with an SIS.

Preferably, ligament repair using Mg-based implant 10 of the present disclosure will be able to restore initial anterior-posterior joint stability and in-situ force in the ACL 15 or other ligament close to normal and better than previously-used suture repair techniques.

Testing has shown that use of the ring 10 and methods of the present disclosure has restored Anterior Tibial Translation (ATT) to within 2-8 mm of an intact joint which represents a reduction of ATT of 50% compared to traditional suture repair.

Testing has also shown that In-situ force in the ACL repaired in accordance with the present disclosure is close to normal. Increased in-situ forces can be handled in the ACL with the Mg-based ring compared to suture repair. Also, repairs made according to the present disclosure provide better anterior-posterior joint stability and in-situ force resistance than in ACLs repaired by traditional suturing.

Ring 10 preferably provides mechanical support to ACL by transmitting load between torn ACL stumps 16 and 18 (reduce stress-shielding) for 12-18 weeks. Ring 10 preferably degrades over such time to let the healing ACL tissue take over. Ring 10 preferably is used as “sheath” to protect injury site during healing.

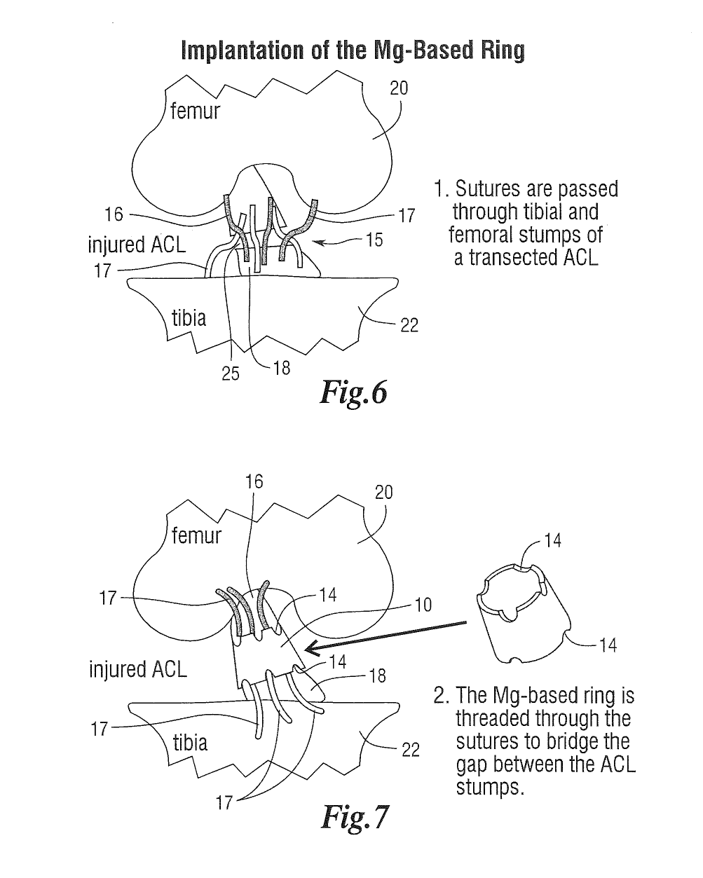

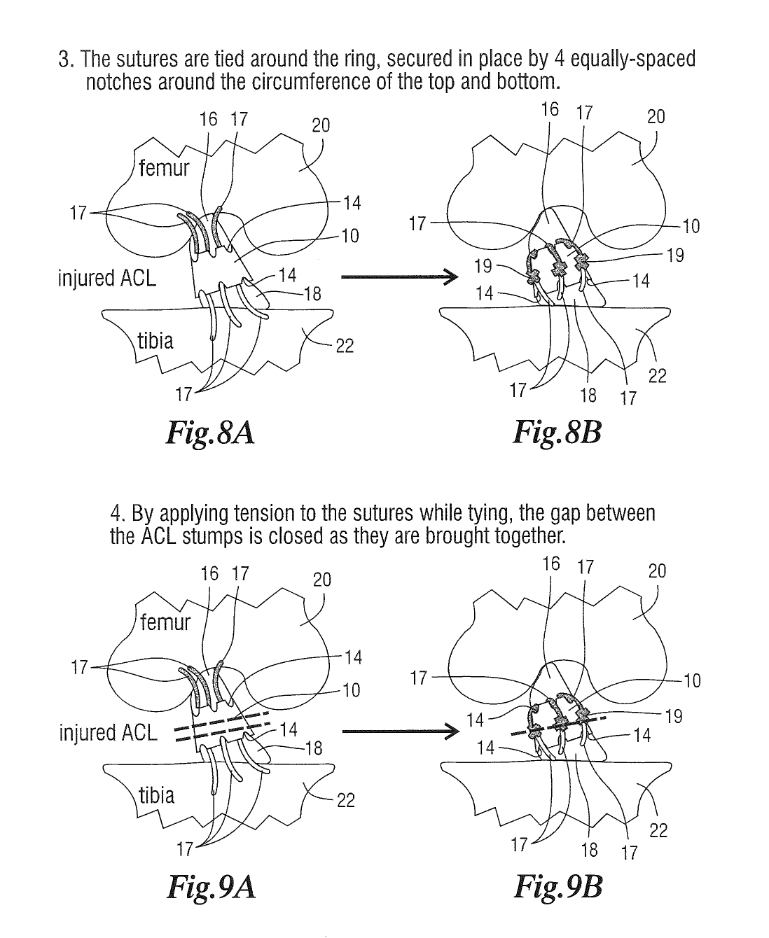

As shown in FIGS. 6-9, in a preferred repair method for a ligament or tendon of the present disclosure, sutures 17 are passed through femoral and femoral stumps, 16 and 18, respectively of a transected ACL 15. As shown in FIG. 6, a preferred Mg-based ring 10 is threaded through the sutures 17 to bridge the gap 25 between the ACL stumps 16 and 18. FIG. 7 shows that preferably, sutures 17 are tied around ring 10 and secured in place around the four equally-spaced notches 14 disposed around the circumference of the top and bottom of ring 10. By applying tension to the sutures 17 while tying, the gap 25 between the ACL stumps 16 and 18 preferably may be closed as shown in FIG. 8. Additionally, the surface of the ring 10 and/or the ligament or tendon 15 preferably may be treated with an ECM such as an SIS or a UBM applied as a coating or sheet wrapper, with or without the ECM's 3-D hydrogel form, to accelerate healing of the ligament or tendon 15. In addition, the hydrogel preferably may be injected into the ligament or tendon 15 instead of being used in a sheet wrapper form. This preferred repair method of the present disclosure using ring 10 with ECM bioscaffolds, hydrogel and/or a preferred suture augmentation technique as shown in FIGS. 6-9 provides for synergistic effects on the healing of ACL 15 or other ligaments or tendons. As shown in FIG. 2, additional suture augmentation preferably may also be employed comprising passing two sutures 23 through tibial and femoral bone tunnels 26 drilled adjacent to the insertion sites and fixing them under manual tension with a titanium button (not shown).

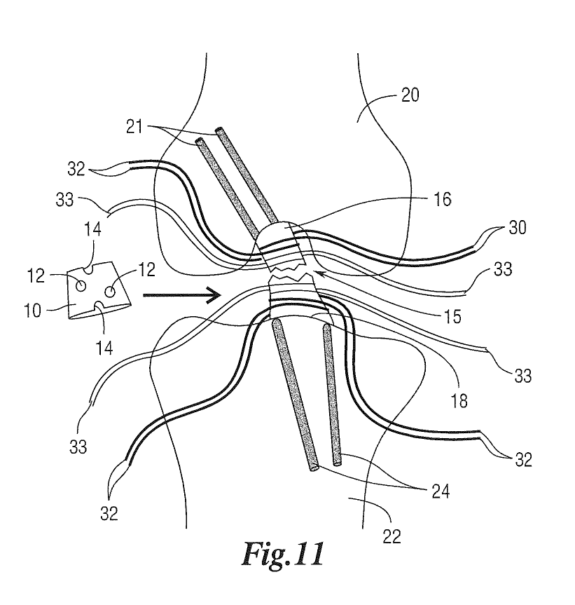

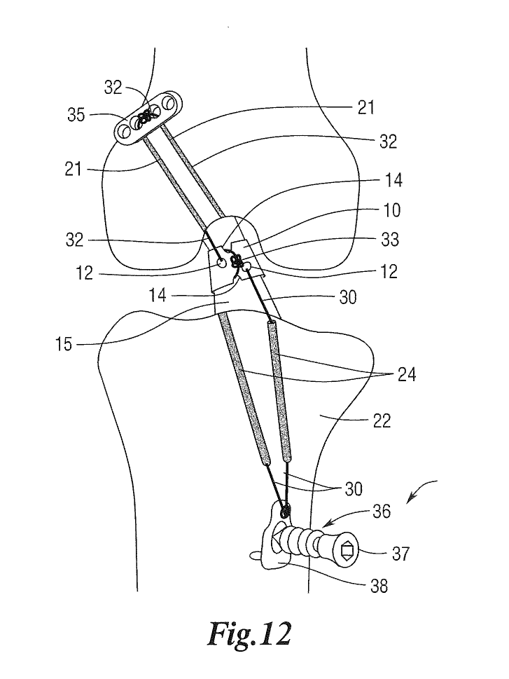

As shown in FIGS. 11-12, in another preferred repair method for a ligament or tendon of the present disclosure, repair sutures 33 and fixation sutures 30, 32 are attached to each respective end of the transected ACL 15. Ring 10 is then threaded onto ACL 15 and repair sutures 33 are tied around notches 14 of ring 10. By applying tension to repair sutures 33 while tying them around notches 14 of ring 10, the gap 25 between the ACL stumps 16 and 18 preferably may be closed such that stumps 16 and 18 contact each other within ring 10.

Preferably, fixation sutures 32 passing through femoral bone tunnels 21 are passed first through and/or around the tibial stump 18, then through the inside of ring 10, then out of ring 10 through holes 12 before going through bone tunnels 21 and being secured to endobutton 35 on femur 20. Fixation sutures 30 passing through tibial bone tunnels 24 are preferably passed first through and/or around femoral stump 16, then through the inside of ring 10, then out of ring 10 through holes 12 before going through bone tunnels 24 and being secured to tibia 22 with a fixation post 37 and double-spiked plate 38.

Additionally, as part of the preferred method shown in FIGS. 11-12, the surface of ring 10 and/or the ligament or tendon 15 preferably may be treated with an ECM such as an SIS or a UBM applied as a coating or sheet wrapper, with or without the ECM's 3-D hydrogel form, to accelerate healing of the ligament or tendon 15. In addition, the hydrogel preferably may be injected into the ligament or tendon 15 instead of being used in a sheet wrapper form. This preferred repair technique of the present disclosure using ring 10 with ECM bioscaffolds, hydrogel and/or a preferred suture augmentation comprising repair sutures 33 and fixation sutures 30, 32 as shown in FIGS. 11-12 provides for synergistic effects on the healing of ACL 15 or other ligaments or tendons.

While the present disclosure has been described in detail and with reference to specific embodiments thereof, it will be apparent to one skilled in the art that various changes and modifications can be made therein without departing from the spirit and scope of the embodiments. Thus, it is intended that the present disclosure cover any such modifications and/or variations provided they come within the scope of the appended claims and their equivalents.

What is claimed is:

1. A method for repairing a ligament or tendon, the method comprising:
passing one or more sutures through and/or around each respective end of a completely or partially torn ligament or tendon;
placing a generally cylindrical hollow body or ring over the respective ends of the completely or partially torn ligament or tendon;
passing the one or more sutures through the generally cylindrical hollow body or ring; and
tying the one or more sutures around or through the generally cylindrical hollow body or ring wherein the sutures are secured in place around or through the generally cylindrical hollow body or ring via one or more notches or holes defined by the generally cylindrical hollow body or ring.

2. The method of claim 1 further comprising tensioning the one or more sutures while tying them around or through the generally cylindrical hollow body or ring.

3. The method of claim 1 further comprising tensioning the one or more sutures while tying them around or through the generally cylindrical hollow body or ring so that the respective ends of the completely or partially torn ligament or tendon are brought into and stay in contact with each other inside the generally cylindrical hollow body or ring after the tying is complete.

4. The method of claim 1 further comprising applying an ECM to the generally cylindrical hollow body or ring.

5. The method of claim 1 wherein the generally cylindrical hollow body or ring comprises a biodegradable material.

6. The method of claim 1 wherein the generally cylindrical hollow body or ring comprises a biodegradable material having an in-situ degradation rate consistent with a healing time of the ligament or tendon.

7. The method of claim 1 wherein the generally cylindrical hollow body or ring comprises magnesium or a magnesium alloy.

8. The method of claim 1 wherein the completely or partially torn ligament comprises an ACL.

9. The method of claim 8 further comprising providing suture augmentation to a joint which the ACL is a part of by passing one or more sutures through each of tibial and femoral bone tunnels disposed adjacent to the joint and securing the sutures to the joint under tension.

10. The method of claim 1 wherein the generally cylindrical hollow body or ring has a first end diameter smaller than a second end diameter and is tapered therebetween.

11. An implant device for use in surgically repairing a ligament or tendon, comprising: a generally cylindrical hollow body, wherein the generally cylindrical hollow body defines a plurality of holes and/or notches for tensioning or tying sutures around or through the generally cylindrical hollow body.

12. The implant device of claim 11 further comprising an ECM.

13. The implant device of claim 11 wherein the generally cylindrical hollow body comprises a biodegradable material.

14. The implant device of claim 11 wherein the generally cylindrical hollow body comprises a biodegradable material having an in-situ degradation rate consistent with a healing time of the ligament or tendon.

15. The implant device of claim 11 wherein the generally cylindrical hollow body comprises magnesium or a magnesium alloy.

16. The implant device of claim 11 wherein the generally cylindrical hollow body has a first end diameter smaller than a second end diameter and is tapered therebetween.
